(19) United States
(12) Patent Application Publication
(10) Pub. No.: US 2009/0047225 A1
(43) Pub. Date: Feb. 19, 2009

(54) STORAGE OF PULVERULENT SUBSTANCES HAVING A HIGH WATER CONTENT

(75) Inventors: Steffen Hasenzahl, Morris Plains, NJ (US); Margarete Drechsler, Gelnhausen (DE); Stephanie Reader, Hanau (DE); Ann Gray, Hanau (DE)

Correspondence Address: OBLON, SPIVAK, MCCLELLAND MAIER & NEUSTADT, P.C., 1940 DUKE STREET, ALEXANDRIA, VA, 22314, US

(73) Assignee: DEGUSSA GmbH, Duesseldorf (DE)

(21) Appl. No.: 11/722,763

(22) PCT Filed: Dec. 23, 2005
(86) PCT No.: PCT/EP05/57160

§ 371 Date: Mar. 7, 2008

(30) Foreign Application Priority Data
Dec. 24, 2004 (DE) 102004062652.9
Nov. 19, 2005 (DE) 102005055226.9

(51) Int. Cl. A61K 8/25; A61Q 17/04; A01N 59/00; A61Q 19/04; A61Q 19/02; C11D 3/39

(52) U.S. Cl. 424/59; 424/724; 424/65; 424/62; 510/375

(57) ABSTRACT

Process to delay the release of water from a pulverulent preparation comprising cosmetic products, cleaning agents and detergents, wherein the pulverulent preparation contains at least 50 wt. % of water and a hydrophobed silicon dioxide powder, wherein the pulverulent preparation is in contact with a hydrophobic material during storage.

The invention concerns a process for the storage of pulverulent substances having a high water content.

“Dry water” is a pulverulent solid having a water content of up to over 95%. It is formed by the intensive mixing of water with hydrophobed pyrogenic silicon dioxide. During this process water droplets are sheathed by the solids particles and prevented from flowing together again. The first experiments on the use of “dry water” as a cosmetic base date from the 1960s (DE-A-1467023, U.S. Pat. No. 3,393,155, Fine Particles series no. 11, Degussa AG). In the late 1990s the concept was revisited by Japanese cosmetics companies in particular and developed further (EP-A-855177, EP-A-1206928, EP-A-1235554). To date, however, only a few cosmetics products have been able to be brought to market.

“Dry water” is produced by the intensive mixing of hydrophobed, pyrogenic silicon dioxide with water. During this process, water droplets produced during the mixing process are sheathed by the non-water-wettable silicon dioxide particles and prevented from flowing together again. “Dry water” can therefore be thought of as an aerosol stabilised by solids particles (cf. the schematic view in FIG. 3). Depending on the mixing conditions, the water droplets are between ten and a few hundred micrometres in size (cf. FIG. 4). Critical to the formation of “dry water” is the hydrophobicity of the pyrogenic silicon dioxide, since it does not disperse in the water phase but is simply “adsorbed” at the surface of the water droplets. Fine-particle water-wettable solids, on the other hand, are distributed in the water phase on mixing, forming a suspension.

In addition to “dry water”, a “dry hydrogen peroxide” is also known. 5-10 wt. % of a hydrophobed silicon dioxide powder is sufficient to produce a fine, free-flowing powder from a hydrogen peroxide solution of variable concentration (WO2004104154).

With regard to its safety characteristics, the product, like other peroxides, has been investigated in terms of its stability and its tendency towards spontaneous decomposition. “Dry hydrogen peroxide” is a storable substance with good handling properties. The active ingredient hydrogen peroxide can be selectively released again by appropriate measures. Mechanical intervention such as grinding, pressure or vacuum lead to the rapid expulsion of the active ingredient from the powder. After stirring the product into an aqueous solution, H₂O₂ is eluted within minutes to hours, depending on the thoroughness of mixing. Dry hydrogen peroxide can be used where the use of liquid H₂O₂ is unfavourable or impossible. For example, it can be used as a bleaching component in cleaning agents and stain removers, or in cosmetics products in accordance with the areas of application and maximum permissible concentrations in the finished cosmetic product regulated by the ordinance on cosmetic products (German cosmetics ordinance). In the same way, the disinfectant action of hydrogen peroxide can be utilised in products for the cosmetics industry or in household products. Applications which make use of the delayed release of the active ingredient under suitable conditions are also conceivable.

The properties and stability of “dry water” and “dry hydrogen peroxide” are determined by various parameters, such as e.g. the hydrophobicity and specific surface area of the silicon dioxide powder, additives to the water phase such as gel forming agents, humectants, solvents, etc., and the production process.

In the production of “dry water”, sufficiently small water droplets and silicon dioxide particles must be produced simultaneously. This requires high mixing energies. Very good results have been obtained for example with a high-speed mixer at speeds of around 10,000 rpm. A conventional kitchen mixer also provided stable powders.

The production of “dry water” generally takes a few seconds to at most a few minutes. The optimum mixing times substantially depend on the composition of the water phase and the chosen mixer. They must therefore be adjusted for each individual case.

EP-A-1386599 discloses a positive effect on the storage stability of “dry water” which occurs when the surfaces of the container with which the dry water comes into contact during its production are hydrophobic. Even this process does not lead to markedly improved storage stabilities, however. Rather, water is separated out of the pulverulent product during storage and an additional separate water phase is formed.

The object of the present invention was therefore to provide a process by which the storage stability of dry water and dry hydrogen peroxide can be improved.

Surprisingly it was found that the object can be achieved by a process to delay the release of water from a pulverulent preparation comprising cosmetic products, cleaning agents and detergents, wherein the pulverulent preparation contains at least 50 wt. % of water and a hydrophobed silicon dioxide powder, which is characterised in that the pulverulent preparation is in contact with a hydrophobic material during storage.

The invention is surprising because according to the teaching from EP-A-1386599 the person skilled in the art would have regarded a hydrophobic walling as being of relevance only in the production of the pulverulent preparation.

By contrast, in the present invention production of the pulverulent preparation can take place in containers having non-hydrophobic walling. Substantial to the lengthening of the storage stability is the subsequent storage in containers in which the pulverulent preparation is in contact with hydrophobic walling only.

There is no restriction on the type of hydrophobed silicon dioxide powder, provided that when it is added to water a pulverulent product is formed. The hydrophobed silicon dioxide powders can preferably be silanised. Halosilanes, alkoxysilanes, silazanes and/or siloxanes can be used for silanisation.

In particular, the following substances can be used as halosilanes:

Organohalosilanes of the type X₃Si(CₙH₂ₙ₊₁) where X=Cl, Br and n=1-20,

Organohalosilanes of the type X₂(R′)Si(CₙH₂ₙ₊₁) where X=Cl, Br and R′=alkyl, n=1-20

Organohalosilanes of the type X(R′)₂Si(CₙH₂ₙ₊₁) where X=Cl, Br, R′=alkyl, n=1-20

Organohalosilanes of the type X₃Si(CH₂)ₘ—R′ where X=Cl, Br, m=0.1-20, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OOC(CH₃)C═CH₂, —OCH₂—CH(O)CH₂, —NH—COO—CH₃, —NH—COO—CH₂—CH₃, —NH—(CH₂)₃Si(OR)₃, —Sₓ—(CH₂)₃Si(OR)₃,

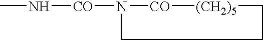

Organohalosilanes of the type (R)X₂Si(CH₂)ₘ—R′ where X=Cl, Br, R=Alkyl, m=0.1-20, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OOC(CH₃)C═CH₂, —OCH₂—CH(O)CH₂, —NH—COO—CH₃, —NH—COO—CH₂—CH₃, —NH—(CH₂)₃Si(OR)₃, —NH—COO—CH₃, —NH—COO—CH₂—CH₃, —NH—(CH₂)₃Si(OR)₃, —Sₓ—(CH₂)₃Si(OR)₃

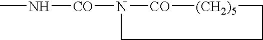

Organohalosilanes of the type (R)₂X Si(CH₂)ₘ—R′ where X=Cl, Br, R=alkyl, m=0.1-20, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OOC(CH₃)C═CH₂, —OCH₂—CH(O)CH₂, —NH—COO—CH₃, —NH—COO—CH₂—CH₃, —NH—(CH₂)₃Si(OR)₃, —Sₓ—(CH₂)₃Si(OR)₃,

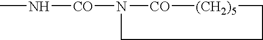

In particular, the following substances can be used as alkoxysilanes:

Organosilanes of the type (RO)₃Si(CₙH₂n+1) where R=alkyl, n=1-20

Organosilanes of the type R′ₓ(RO)_{y}Si(CₙH₂ₙ₊₁) where R=alkyl, R′=alkyl, n=1-20, x+y=3, x=1, 2, y=1, 2

Organosilanes of the type (RO)₃Si(CH₂)ₘ—R′ where R=alkyl, m=0.1-20, R′=alkyl, aryl (for example —C₆H₅), —C₄F₉, OCF₂—CHF—CF₃, —C₆F₁₃, —O—CF₂—CHF₂, —NH₂, —N₃, —SCN, —CH═CH₂, —OOC(CH₃)C═CH₂, —OCH₂—CH(O)CH₂, —NH—COO—CH₃, —NH—COO—CH₂—CH₃, —NH—(CH₂)₃Si(OR)₃, —Sₓ—(CH₂)₃Si(OR)₃

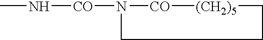

Organosilanes of the type (R″)ₓ(RO)_{y}Si(CH₂)ₘ—R′ where R″=alkyl, x+y=2, x=1, 2, y=1, 2, R′=alkyl, aryl (for example —C₆H₅), —C₄F₉, —OCF₂—CHF—CF₃, —C₆F₁₃, —O—CF₂—CHF₂, —NH₂, —N₃, —SCN, —CH═CH₂, —OOC(CH₃)C═CH₂, —OCH₂—CH(O)CH₂, NH—COO—CH₃, —NH—COO—CH₂—CH₃, —NH—(CH₂)₃Si(OR)₃, —Sₓ—(CH₂)₃Si(OR)₃

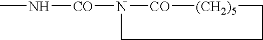

Trimethoxyoctyl silane [(CH₃O)₃—Si—C₈H₁₇] (e.g. DYNASYLAN® OCTMO, Degussa AG) can preferably be used as silanising agent.

In particular, the following substances can be used as silazanes:

Silazanes of the type:

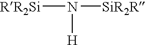

where R=alkyl, R′=alkyl, vinyl, and e.g. hexamethyldisilazane (for example DYNASYLAN® HMDS).

In particular, the following substances can be used as siloxanes:

Cyclic polysiloxanes of the type D 3, D 4, D 5, for example octamethyl cyclotetrasiloxane=D 4

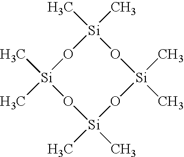

Polysiloxanes or silicone oils of the type:

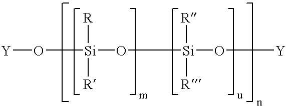

R=alkyl, aryl, (CH₂)ₙ—NH₂, H
R′=alkyl, aryl, (CH₂)ₙ—NH₂, H
R″=alkyl, aryl, (CH₂)ₙ—NH₂, H
R′″=alkyl, aryl, (CH₂)ₙ—NH₂, H
Y=CH₃, H, CₙH₂ₙ₊₁ where n=1-20
Y=Si(CH₃)₃, Si(CH₃)₂H
   Si(CH₃)₂OH, Si(CH₃)₂(OCH₃)
   Si(CH₃)₂(CₙH₂ₙ₊₁) where n=1-20
m=0, 1, 2, 3, . . . ∞
n=0, 1, 2, 3, . . . ∞
u=0, 1, 2, 3, . . . ∞

Silanisation can be performed by spraying the silicon dioxide powder with the silanising agent, which can optionally be dissolved in an organic solvent, such as ethanol for example, and then heat treating the mixture at a temperature of 105 to 400° C. for a period of 1 to 6 h.

There is no restriction on the silicon dioxide powders used for hydrophobing. Silicon dioxide powders of pyrogenic origin can preferably be used. The term pyrogenic here covers powders which are obtainable by flame oxidation or flame hydrolysis from suitable silicon compounds. As a rule, silicon tetrachloride is hydrolysed in a hydrogen and oxygen flame to give silicon dioxide.

Suitable commercially available hydrophobed silicon dioxide powders can be Aerosil® R106, R202, R805, R812, R812S, R8200, R812 VV60, R812 VV90, R812S VV60, R812S VV90, R104 V, R202 VV90, R805 VV90 (Degussa) or HDK® H2000, H2050, H3004 (Wacker).

In the process according to the invention, the pulverulent preparation as a cosmetic product can contain a substance from the group comprising
a) UV light screening filters,
b) Dyes and pigments,
c) Humectants/skin moisturising agents,
d) Deodorising and antiperspirant agents,
e) Biogenic substances,
f) Insect repellent agents,
g) Hydrotropes,
h) Anti-dandruff agents,
i) Bleaching or skin lightening agents and self-tanning agents,
j) Preservatives,
k) Surfactants/emulsifiers,
l) Perfume oils and/or plant extracts.

### a) UV Light Screening Filters

UV light screening filters according to the invention are organic substances (light screening filters) which are liquid or crystalline at room temperature and which are capable of absorbing ultraviolet rays and of giving off the absorbed energy again in the form of longer-wave radiation, for example heat. UV filters can be oil-soluble or water-soluble. Examples of oil-soluble substances that can be cited are:
3-benzylidene camphor or 3-benzylidene norcamphor and derivatives thereof, for example 3-(4-methyl benzylidene) camphor as described in EP 0 693 471 B1
4-aminobenzoic acid derivatives, preferably 4-(dimethylamino)benzoic acid-2-ethylhexyl ester, 4-(dimethylamino)benzoic acid-2-octyl ester and 4-(dimethylamino)benzoic acid amyl ester
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (octocrylene)
esters of salicylic acid, preferably salicylic acid-2-ethylhexyl ester, salicylic acid-4-isopropyl benzyl ester, salicylic acid homomenthyl ester
derivatives of benzophenone, preferably 2-hydroxy-4-methoxybenzophenone, 2-hydroxy-4-methoxy-4′-methyl benzophenone, 2,2′-dihydroxy-4-methoxybenzophenone
esters of benzalmalonic acid, preferably 4-methoxybenzalmalonic acid di-2-ethylhexyl ester
triazine derivatives, such as e.g. 2,4,6-trianilino-(p-carbo-2′-ethyl-1′-hexyloxy)-1,3,5-triazine and octyl triazone, as described in EP 0 818 450 A1 or dioctyl butamido triazone (Uvasorb™ HEB)
propane-1,3-diones, such as e.g. 1-(4-tert-butylphenyl)-3-(4′-methoxyphenyl)propane-1,3-dione
ketotricyclo(5.2.1.0)decane derivatives, as described in EP 0 694 521 B1.

Suitable water-soluble substances include:
2-phenyl benzimidazole-5-sulfonic acid and alkali, alkaline-earth, ammonium, alkyl ammonium, alkanol ammonium and glucammonium salts thereof
sulfonic acid derivatives of benzophenones, preferably 2-hydroxy-4-methoxybenzophenone-5-sulfonic acid and salts thereof
sulfonic acid derivatives of 3-benzylidene camphor, such as e.g. 4-(2-oxo-3-bornylidene methyl) benzenesulfonic acid and 2-methyl-5-(2-oxo-3-bornylidene)sulfonic acid and salts thereof.

Typical examples of UV-A filters are in particular derivatives of benzoyl methane, such as e.g. 1-(4′-tert-butylphenyl)-3-(4′-methoxyphenyl)propane-1,3-dione, 4-tert-butyl-4′-methoxydibenzoylmethane (Parsol™ 1789), 1-phenyl-3-(4′-isopropylphenyl)-propane-1,3-dione and enamine compounds, as described in DE 191 12 033 A1 (BASF). The UV-A and UV-B filters can naturally also be used in mixtures. Particularly favourable combinations consist of derivatives of benzoyl methane, for example 4-tert-butyl-4′-methoxydibenzoyl methane (Parsol™ 1789) and 2-cyano-3,3-phenyl cinnamic acid-2-ethylhexyl ester (octocrylene) in combination with esters of cinnamic acid, preferably 4-methoxycinnamic acid-2-ethylhexyl ester and/or 4-methoxycinnamic acid propyl ester and/or 4-methoxycinnamic acid isoamyl ester. Such combinations are advantageously combined with water-soluble filters, such as e.g. 2-phenyl benzimidazole-5-sulfonic acid and alkali, alkaline-earth, ammonium, alkyl ammonium, alkanol ammonium and glucammonium salts thereof.

UV filters which can be dissolved or emulsified in the aqueous phase are particularly advantageous.

In addition to the cited soluble substances, insoluble light screening pigments, namely finely dispersed metal oxide powders, which are preferably in hydrophobed form, or salts, are also suitable for this purpose. Examples of suitable metal oxide powders or hydrophobed metal oxide powders can be titanium dioxide powder, aluminium oxide powder, zinc oxide powder and/or a mixed oxide powder with the elements Si, Ti, Al, Zn, Fe, B, Zr and/or Ce.

The proportion of metal oxide powders, based on the sum of hydrophobed silicon dioxide powder and metal oxide powder, is preferably less than 50 wt. % and particularly preferably less than 30 wt. %.

Silicates (talc), barium sulfate or zinc stearate can also be used.

So-called micropigments or nanopigments are preferably used in sunscreens. The particles should have an average diameter of less than 100 nm, preferably between 5 and 50 nm and in particular between 15 and 30 nm. They can have a spherical form, but such particles having an ellipsoid form or other form deviating from the spherical shape can also be used.

Typical examples are coated titanium dioxides such as e.g. UV-titanium M212, M 262 and X 111 (Kemira), AEROXIDE TiO2 P25, PF2, T 805 and T 817 (Degussa), micro titanium dioxide MT-150 W, MT-100 AQ, MT-100 SA, MT-100 HD, MT-100 TV (Tayca), Eusolex™ T2000 (Merck), zinc oxide neutral H&R and zinc oxide NDM (Haarmann & Reimer) and Z-Cote and Z-Cote HP1 (BASF). Dispersions such as TEGO Sun TAQ 40, a 40 wt. % aqueous dispersion of a hydrophobed titanium dioxide (Degussa) can also be used. Other suitable UV light screening filters can be found in the survey by P. Finkel in SÖFW-Journal 122, 543 (1996) and Parf. Kosm. 3, 11 (1999). Optical brighteners such as e.g. 4,4′-diaminostilbene-2,2′-disulfonic acid and derivatives thereof can also be used.

In addition to the two groups of primary light screening agents cited above, secondary light screening agents of the antioxidant type can also be used, which interrupt the photochemical reaction chain that is initiated when UV radiation penetrates the skin. Typical examples of these are amino acids (for example glycine, histidine, tyrosine, tryptophane) and derivatives thereof, imidazoles (for example urocanic acid) and derivatives thereof, peptides such as D,L-carnosine, D-carnosine, L-carnosine and derivatives thereof (for example anserine), carotenoids, carotenes (for example alpha-carotene, beta-carotene, lycopene) and derivatives thereof, chlorogenic acid and derivatives thereof, lipoic acid and derivatives thereof (for example dihydrolipoic acid), aurothioglucose, propyl thiouracil and other thiols (for example thioredoxine, glutathione, cysteine, cystine, cystamine and glycosyl, N-acetyl, methyl, ethyl, propyl, amyl, butyl and lauryl, palmitoyl, oleyl, gamma-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example buthionine sulfoximines, homocysteine sulfoximine, buthionine sulfones, penta-, hexa-, heptathionine sulfoximine) in very small tolerated doses (for example pmol to mu mol/kg), also (metal) chelators (for example alpha-hydroxy fatty acids, palmitic acid, phytic acid, lactoferrin), alpha-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example gamma-linolenic acid, linoleic acid, oleic acid), folic acid and derivatives thereof, ubiquinone and ubiquinol and derivatives thereof, vitamin C and derivatives (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoic resin, rutinic acid and derivatives thereof, alpha-glycosyl rutin, ferulic acid, furfurylidene glucitol, carnosine, butyl hydroxytoluene, butyl hydroxyanisole, nordihydroguaiac acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and the derivatives (salts, esters, ethers, sugars, nucleotides, nucleosides, peptides and lipids) of these cited active ingredients that are suitable according to the invention.

### b) Dyes and Pigments

Dyes which can be used are natural, vegetable or animal dyes such as, for example, betanin, bixin, carmine, carotene, chlorophyll, sepia etc. and derivatives thereof, as well as synthetic organic dyes, such as e.g. azo, anthraquinone, triphenylmethane dyes, etc. Dyes that are water-soluble or are dispersible in water can be particularly preferred.

The pulverulent preparation can also contain inorganic pigments, such as ochre, umber, red bole, sienna, chalk, etc. and synthetic inorganic pigments such as iron oxides, ultramarines, titanium dioxide, zinc oxide, mica-based pigments, such as e.g. pearlescent pigments. Water-wettable pigments can be particularly preferred.

### c) Humectants/Skin Moisturising Agents

The pulverulent preparation can also contain humectants. These serve to further optimise the sensory properties of the composition and to regulate the moisture of the skin. At the same time the low-temperature stability of the preparations according to the invention, especially in the case of emulsions, is increased. The humectants are conventionally included in an amount of 0.1 to 15 wt. %, preferably 1 to 10 wt. %, and in particular 5 to 10 wt. %.

Suitable examples are, inter alia, amino acids, pyrrolidone carboxylic acid, lactic acid and salts thereof, lactitol, urea and urea derivatives, uric acid, glucosamine, creatinine, breakdown products of collagen, chitosan or chitosan salts/derivatives, and in particular polyols and polyol derivatives (for example glycerol, diglycerol, triglycerol, ethylene glycol, propylene glycol, butylene glycol, pentylene glycol, erythritol, 1,2,6-hexanetriol, polyethylene glycols such as PEG-4, PEG-6, PEG-7, PEG-8, PEG-9, PEG-10, PEG-12, PEG-14, PEG-16, PEG-18, PEG-20), sugars and sugar derivatives (inter alia fructose, glucose, maltose, maltitol, mannitol, inositol, sorbitol, sorbityl silanediol, sucrose, trehalose, xylose, xylitol, glucuronic acid and salts thereof), ethoxylated sorbitol (Sorbeth-6, Sorbeth-20, Sorbeth-30, Sorbeth-40), honey and hydrogenated honey, hydrogenated starch hydrolysates and mixtures of hydrogenated wheat protein and PEG-20-acetate copolymer. The preparation according to the invention can particularly preferably contain glycerol, diglycerol, triglycerol and butylene glycol.

### d) Deodorising and Antiperspirant Agents

Deodorising and antiperspirant agents can also be added to the pulverulent preparation. These active ingredients include astringent metal salts (antiperspirant agents), germ-inhibiting agents, enzyme inhibitors, odour absorbers, odour maskers or any combination of these active ingredients. The deodorising/antiperspirant agents can be included in the pulverulent preparation in an amount from 0.1 to 30 wt. %, preferably 5 to 25 wt. % and in particular 10 to 25 wt. % (based on the amount of preparation).

Aluminium chlorohydrates, aluminium zirconium chlorohydrates and zinc salts, for example, can be used as antiperspirant agents. In addition to the chlorohydrates, the preparation according to the invention can also contain aluminium hydroxylactates and acid aluminium/zirconium salts, for example Locron™ (formula [Al₂(OH)₅Cl]×2.5 H₂O, Clariant GmbH) or Rezal™ 36G (aluminium zirconium tetrachlorohydrex glycine complexes, Reheis).

Enzyme inhibitors, for example esterase inhibitors, can be added as additional deodorising agents. These are preferably trialkyl citrates, such as trimethyl citrate, tripropyl citrate, triisopropyl citrate, tributyl citrate and in particular triethyl citrate (Hydagen™ C. A. T., Cognis Deutschland GmbH). The substances inhibit the enzyme activity of sweat-destroying bacteria, thereby reducing the formation of odours. Other substances that can be considered as esterase inhibitors are sterol sulfates or phosphates, such as e.g. lanosterol, cholesterol, campesterol, stigmasterol and sitosterol sulfate or phosphate, dicarboxylic acids and esters thereof, such as e.g. glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, such as e.g. citric acid, malic acid, tartaric acid or tartaric acid diethyl ester. Antibacterial agents, which influence microbial flora and kill or inhibit the growth of sweat-destroying bacteria, can likewise be included in the preparation according to the invention. Examples of these are chitosan, phenoxyethanol, chlorohexidine gluconate or 5-chloro-2-(2,4-dichlorophenoxy) phenol (Irgasan™, Ciba-Geigy, Basle/CH).

All substances that are active against gram-positive bacteria are suitable in principle as germ-inhibiting agents, such as e.g. 4-hydroxybenzoic acid and its salts and esters, N-(4-chlorophenyl)-N′-(3,4 dichlorophenyl) urea, 2,4,4′-trichloro-2′-hydroxydiphenyl ether (triclosan), 4-chloro-3,5-dimethylphenol, 2,2′-methylene bis-(6-bromo-4-chlorophenol), 3-methyl-4-(1-methylethyl) phenol, 2-benzyl-4-chlorophenol, 3-(4-chlorophenoxy)-1,2-propanediol, 3-iodine-2-propynyl butyl carbamate, chlorohexidine, 3,4,4′-trichlorocarbanilide (TTC), antibacterial perfumes, thymol, thyme oil, eugenol, clove oil, menthol, mint oil, farnesol, phenoxyethanol, glycerol monocaprinate, glycerol monocaprylate, glycerol monolaurate (GML), diglycerol monocaprinate (DMC), salicylic acid-N-alkylamides, such as e.g. salicylic acid-n-octylamide or salicylic acid-n-decylamide.

Substances that can absorb and largely retain odour-producing compounds are suitable as odour absorbers. They lower the partial pressure of the individual components, thereby also reducing their speed of propagation. It is important here that perfumes are not adversely affected. As their main component, for example, they contain a complex zinc salt of ricinoleic acid or special, largely odour-neutral aromatics, which are known to the person skilled in the art as “fixatives”, such as e.g. extracts of labdanum or styrax or certain abietic acid derivatives.

Fragrance substances or perfume oils act as odour maskers which, in addition to their function as odour maskers, give the deodorants their scent. Examples of perfume oils that can be cited by way of example are mixtures of natural and synthetic fragrance substances. Natural fragrance substances are extracts of flowers, stems and leaves, fruits, fruit skins, roots, woods, herbs and grasses, needles and twigs as well as resins and balsams. Animal raw materials are also suitable, such as e.g. civet and castoreum. Typical synthetic fragrance compounds are products of the ester, ether, aldehyde, ketone, alcohol or hydrocarbon type. Fragrance compounds of the ester type are for example benzyl acetate, p-tert-butyl cyclohexyl acetate, linalyl acetate, phenyl ethyl acetate, linalyl benzoate, benzyl formate, allyl cyclohexyl propionate, styrallyl propionate and benzyl salicylate. The ethers include, for example, benzyl ethyl ethers, the aldehydes include, for example, the linear alkanals having 8 to 18 carbon atoms, citral, citronellal, citronellyl oxyacetaldehyde, cyclamen aldehyde, hydroxy citronellal, lilial and bourgeonal, the ketones include the ionones and methyl cedryl ketone, the alcohols include anethol, citronellol, eugenol, isoeugenol, geraniol, linalool, phenylethyl alcohol and terpineol, the hydrocarbons primarily include the terpenes and balsams. Mixtures of various fragrance substances are preferably used, however, which together produce a pleasant scent. Essential oils of relatively low volatility, which are mainly used as aroma components, are also suitable as perfume oils, for example sage oil, camomile oil, clove oil, melissa oil, mint oil, cinnamon leaf oil, lime-blossom oil, juniper berry oil, vetiver oil, olibanum oil, galbanum oil, labdanum oil and lavandin oil. Bergamot oil, dihydromyrcenol, lilial, lyral, citronellol, phenyl ethyl alcohol, alpha-hexylcinnamaldehyde, geraniol, benzyl acetone, cyclamen aldehyde, linalool, boisambrene forte, ambroxan, indole, hedione, sandelice, lemon oil, mandarin oil, orange oil, allyl amyl glycolate, cyclovertal, lavandin oil, clary sage oil, beta-damascone, geranium oil bourbon, cyclohexyl salicylate, Vertofix Coeur, Iso-E-Super, Fixolide NP, evernyl, iraldein gamma, phenyl acetic acid, geranyl acetate, benzyl acetate, rose oxide, romilat, irotyl and floramat are preferably used, alone or in mixtures.

### e) Biogenic Substances

Suitable biogenic active ingredients are for example tocopherol, tocopherol acetate, tocopherol palmitate, ascorbic acid, (deoxy)ribonucleic acid and fragmentation products thereof, beta-glucane, retinol, bisabolol, allantoin, phytantriol, panthenol, panthotenic acid, fruit acids, alpha-hydroxy acids, amino acids, ceramides, pseudoceramides, essential oils, plant extracts, such as e.g. prunus extract, bambara nut extract, and vitamin complexes.

### f) Insect Repellent Agents

A pulverulent preparation can additionally contain at least one insect repellent agent or a combination of these agents. Suitable insect repellents include, for example, N,N-diethyl-m-toluamide, 1,2-pentanediol or 3-(N-n-butyl-N-acetyl amino) propionic acid ethyl ester)(Insect Repellent 3535, Merck KGaA) and butyl acetyl aminopropionates. They are conventionally present in an amount of 0.1-10 wt. %, preferably 1-8 wt. % and particularly preferably in an amount of 2-6 wt. %, based on the preparation.

### g) Hydrotropes

A pulverulent preparation can contain hydrotropes, such as e.g. ethanol, isopropyl alcohol, or polyols. Polyols for consideration here preferably contain 2 to 15 carbon atoms and at least two hydroxyl groups. The polyols can also contain other functional groups, in particular amino groups, or be modified with nitrogen. Typical examples are:
glycerol
alkylene glycols, such as e.g. ethylene glycol, diethylene glycol, propylene glycol, butylene glycol, pentylene glycol, hexylene glycol and polyethylene glycols having an average molecular weight of 100 to 1000 daltons
technical oligoglycerol mixtures having an intrinsic degree of concentration of 1.5 to 10, such as e.g. technical diglycerol mixtures having a diglycerol content of 40 to 50 wt. %
methylol compounds, such as in particular trimethylol ethane, trimethylol propane, trimethylol butane, pentaerythritol and dipentaerythritol
short-chain alkyl glucosides, in particular those having 1 to 8 carbons in the alkyl radical, such as e.g. methyl and butyl glucoside
sugar alcohols having 5 to 12 carbon atoms, such as sorbitol or mannitol, for example
sugars having 5 to 12 carbon atoms, such as glucose or sucrose, for example
amino sugars, such as glucamine for example
dialcohol amines, such as diethanolamine or 2-amino-1,3-propanediol.

### h) Anti-Dandruff Agents

Suitable anti-dandruff agents in the pulverulent preparation include piroctone olamine (1-hydroxy-4-methyl-6-(2,4,4-trimethylpentyl)-2-(1H)-pyridinone monoethanolamine salt), Baypival™ (climbazole), Ketoconazol™, (4-acetyl-1-4-[2-(2,4-dichlorophenyl)-r-2-(1-imidazol-1-yl methyl)-1,3-dioxylan-c-4-ylmethoxyphenyl piperazine, ketoconazole, elubiol, selenium disulfide, colloidal sulfur, sulfur polyethylene glycol sorbitane monooleate, sulfur ricinol polyethoxylate, sulfur tar distillate, salicylic acid (or in combination with hexachlorophene), undecylenic acid monoethanolamide sulfosuccinate Na salt, Lamepon™ UD (protein undecylenic acid condensate), zinc pyrithione, aluminium pyrithione and magnesium pyrithione/dipyrithione magnesium sulfate.

### i) Bleaching or Skin Lightening Agents and Self-Tanning Agents

A pulverulent preparation can contain bleaching or skin lightening agents, such as e.g. basic bismuth salts, hydroquinone, oxygen-eliminating compounds, such as e.g. zinc peroxide, urea peroxide, hydrogen peroxide and/or organic peroxides. The pulverulent preparation can particularly preferably contain hydrogen peroxide, which is used in the form of aqueous solutions. Suitable examples of tyrosinase inhibitors, which prevent the formation of melanin and are used in depigmenting agents, include arbutin, ferulic acid, kojic acid, coumarinic acid and ascorbic acid (vitamin C, sodium ascorbyl phosphate, magnesium ascorbyl phosphate). Particularly suitable is Cosmocair C 250 from Degussa AG.

Dihydroxyacetone, for example, is suitable as a self-tanning agent.

### j) Preservatives

Suitable preservatives are, for example, phenoxyethanol, formaldehyde solution, parabens, pentane diol or sorbic acid and the silver complexes known under the name Surfacine™ and the other classes of substances listed in Annex 6, Part A and B of the German cosmetics ordinance.

### k) Surfactants/Emulsifiers

The pulverulent preparation can contain surfactants/emulsifiers. The amount of these substances in the preparation is critical, however, since their wetting behaviour can prevent the formation of a powder when hydrophobed silicon dioxide powder is added, so that a pulverulent preparation cannot be obtained. The pulverulent preparations therefore generally contain no surfactants/emulsifiers. There is no restriction on the type of surfactant/emulsifier. A pulverulent preparation can thus contain non-ionic, zwitterionic, amphoteric, cationic and also anionic surfactants.

### l) Perfume Oils and Plant Extracts

The pulverulent preparation can contain perfume oils. These can be natural, plant and animal as well as synthetic fragrance substances or mixtures thereof. Natural fragrance substances are obtained inter alia by extraction from flowers, stems, leaves, fruit, fruit skins, roots and resins of plants. Animal raw materials are also suitable, such as e.g. civet and castoreum. Typical synthetic fragrance compounds are products of the ester, ether, aldehyde, ketone, alcohol or hydrocarbon type. Mixtures of various fragrance substances are preferably used, which together produce an attractive perfume note.

Plant extracts that can be used include, for example, extracts of arnica, birch, camomile, burr root, beard lichen, poplar, stinging nettle and walnut shells.

### m) Active Ingredients

The pulverulent preparation can contain hormones such as e.g. oxytocin, corticotropin, vasopressin, secretin, gastrin.

The pulverulent preparation can also contain hydrophobic, organic powders of polystyrenes, polyethylenes, organopolysiloxanes, polymethyl silsesquioxanes, N-acyl-lysine, polyethylene tetrafluoride resins, acrylic acid resins, epoxy resins, polymethyl methacrylates, acrylonitrile-methacrylate copolymers, vinylidene chloride-methacrylic acid copolymers and/or nylon powder.

The pulverulent preparation can contain hydrophobed inorganic metal oxide powders, such as e.g. titanium dioxide or aluminium oxide, wherein these metal oxide powders can be of pyrogenic origin. The proportion of these hydrophobed metal oxide powders is preferably less than 50 wt. % and particularly preferably less than 30 wt. %, based in each case on the total amount of hydrophobed silicon dioxide powder and hydrophobed metal oxide powder.

Pulverulent preparations containing viscosity regulators can also be used in the process according to the invention. These can preferably be:
hydrogel formers or hydrocolloids, such as e.g. modified polysaccharides such as cellulose ethers and cellulose esters, for example carboxymethyl cellulose, methyl cellulose, hydroxyethyl cellulose, methylhydroxyethyl cellulose, methylhydroxypropyl cellulose, xanthan gum, guar guar, agar agar, alginates and tyloses;
inorganic hydrocolloids such as bentonites, magnesium aluminium silicates, silicon dioxide; and synthetic hydrocolloids such as polyacrylates (for example Carbopole™ and Pemulen types from Goodrich; Synthalene™ from Sigma; Keltrol types from Kelco; Sepigel types from Seppic; Salcare types from Allied Colloids), uncrosslinked and polyol-crosslinked polyacrylic acids, polyacrylamides, polyvinyl alcohol and polyvinyl pyrrolidone.

Surfactants such as e.g. ethoxylated fatty acid glycerides, esters of fatty acids with polyols, such as e.g. pentaerythritol or trimethylol propane, fatty alcohol ethoxylates with concentrated homologue distribution, alkyl oligoglucosides and electrolytes, such as e.g. common salt and ammonium chloride, can also be used for viscosity regulation.

Also suitable as viscosity regulators are anionic, zwitterionic, amphoteric and nonionic copolymers, such as e.g. vinyl acetate/crotonic acid copolymers, vinyl pyrrolidone/vinyl acrylate copolymers, vinyl acetate/butyl maleate/isobornyl acrylate copolymers, methyl vinyl ether/maleic anhydride copolymers and esters thereof, acrylamidopropyl trimethyl ammonium chloride/acrylate copolymers, octyl acrylamide/methyl methacrylate/tert-butyl aminoethyl methacrylate/2-hydroxypropyl methacrylate copolymers, vinyl pyrrolidone/vinyl acetate copolymers, vinyl pyrrolidone/dimethyl aminoethyl methacrylate/vinyl caprolactam terpolymers and optionally derivatised cellulose ethers and silicones. Other suitable polymers and thickeners are listed in Cosm. Toil. 108, 95 (1993).

The proportion of viscosity regulator in the pulverulent preparation can be up to 20 wt. %, preferably 1 to 5 wt. %.

The preparation according to the invention can also contain at least one oil body. Oil bodies are understood to be substances or mixtures of substances that are liquid at 20° C. and are immiscible with water at 25° C. The combination with oil bodies allows the sensory properties of the preparations to be optimised.

Examples of oil bodies include guerbet alcohols based on fatty alcohols having 6 to 18, preferably 8 to 10 carbon atoms (for example Eutanol™ G), esters of linear C₆-C₂₂ fatty acids with linear or branched C₆-C₂₂ fatty alcohols or esters of branched C₆-C₁₃ carboxylic acids with linear or branched C₆-C₂₂ fatty alcohols, such as e.g. myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-C₂₂ fatty acids with branched alcohols, in particular 2-ethylhexanol, esters of C₃-C₃₈ alkyl hydroxycarboxylic acids with linear or branched C₆-C₂₂ fatty alcohols—in particular diethylhexyl malate, esters of linear and/or branched fatty acids with polyhydric alcohols (such as e.g. propylene glycol, dimerdiol or trimertriol) and/or guerbet alcohols, triglycerides based on C₆-C₁₀ fatty acids, liquid mono-/di-/triglyceride mixtures based on C₆-C₁₈ fatty acids, esters of C₆-C₂₂ fatty alcohols and/or guerbet alcohols with aromatic carboxylic acids, in particular benzoic acid, esters of C₂-C₁₂ dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂ fatty alcohol carbonates, such as e.g. dicaprylyl carbonates (Cetiol™ CC), guerbet carbonates based on fatty alcohols having 6 to 18, preferably 8 to 10 C atoms, esters of benzoic acid with linear and/or branched C₆-C₂₂ alcohols (for example Finsolv™ TN), linear or branched, symmetrical or asymmetrical dialkyl ethers having 6 to 22 carbon atoms per alkyl group, such as e.g. dicaprylyl ethers (Cetiol™ OE), ring-opening products of epoxidised fatty acid esters with polyols (Hydagen™ HSP, Sovermol™ 750, Sovermol™ 1102), and/or aliphatic or naphthenic hydrocarbons, such as e.g. mineral oil, vaseline, petrolatum, squalane, squalene, dialkyl ethers, dialkyl carbonates and/or dialkyl cyclohexanes.

Pulverulent preparations containing silicone compounds can also be used in the process according to the invention. These can be cyclomethicones, dimethicones, dimethyl polysiloxanes, methyl phenyl polysiloxanes, cyclic silicones, amino-, fatty acid-, alcohol-, polyether-, epoxy-, fluorine-, glycoside- and/or alkyl-modified silicone compounds. Also suitable are simethicones, which are mixtures of dimethicones having an average chain length of 200 to 300 dimethyl siloxane units and silicon dioxide or hydrogenated silicates.

Depending on the application form, the amount of oil bodies in the overall composition can be between 0.1 and 10 wt. %. The amount can particularly preferably vary between 0.5 and 3 wt. %.

In the process according to the invention the pulverulent preparation in the form of a cleaning agent or detergent can contain hydrogen peroxide. The same hydrophobed silicon dioxide powders can be used as described above. They preferably have a methanol wettability of at least 40. The hydrogen peroxide content can preferably be between 10 and 50 wt. %. The proportion of hydrophobed silicon dioxide powder can preferably be less than 9 wt. %, based on the overall mixture. Hydrogen peroxide is used as an aqueous solution, preferably with a proportion of hydrogen peroxide of between 5 and 70 wt. %. Solutions having an H₂O₂ content of 35 and 50 wt. % are usually used. The solutions are advantageously stabilised to prevent decomposition. The nature and amount of stabiliser(s) primarily depends on the proportion of hydrogen peroxide in the aqueous solution.

The hydrophobic material with which the pulverulent preparation is in contact during storage can preferably be polyethylene, polypropylene, polyethylene terephthalate and/or teflon.

### EXAMPLES

### Examples 1 to 6

### Cosmetic Preparation

88.92 g of deionised water and 5.0 g of propylene glycol (Caelo) are placed in a beaker, 0.8 g of LCW Covagel (LCW Sensient) are added whilst stirring with a magnetic stirrer and this mixture is then stirred for a further 15 minutes at room temperature. The clear solution is poured into a suitable 500-ml stainless steel beaker, 5.0 g of the hydrophobed silicon dioxide powder or a mixture of the hydrophobed silicon dioxide powder with a hydrophobed metal oxide powder are added and the components are mixed with a high-speed mixer (DISPERMAT®; VMA-Getzman, disc diameter 5 cm) for 60 seconds at 10,000 rpm. The product obtained is then introduced into 250-ml screw-top bottles made from glass or polyethylene and stored in the tightly closed containers for three months at room temperature. Visual assessment of the preparation after three months is carried out on the basis of the criteria set out in Table 1. Table 2 shows the results of the visual assessment.

### Examples 7 to 11

### Cleaning Agent/Detergent

93.0 g of a 10-percent hydrogen peroxide solution (commercially stabilised) are mixed in a 500-ml stainless steel beaker with 7.0 g of a hydrophobed silicon dioxide powder in a high-speed mixer (DISPERMAT®; VMA-Getzmann, disc diameter 5 cm) for five minutes at 10,000 rpm. The product obtained is introduced into 250-ml screw-top bottles made from glass or polyethylene and stored in the tightly closed containers for three months at room temperature. Visual assessment of the preparation after three months is carried out on the basis of the criteria set out in Table 1. Table 3 shows the results of the visual assessment.

The examples show the clearly improved storage stability of pulverulent preparations when they are in contact with hydrophobic surfaces of containers.

TABLE 1
Assessment of storage stability

| | |
|---|---|
| A | Unchanged, i.e. no separation of water |
| B | Appearance of a few drops of liquid on the lid, |
| | base and/or walls of the container |
| C | Slight separation of water |
| D | Considerable separation of water |

TABLE 2
Storage stability of cosmetic preparations

| | | Storage container | |
|---|---|---|---|
| | | material | |
| Example | Silicon dioxide powder | Glass | Polyethylene |
| 1 | AEROSIL © R 812S | C | A |
| 2 | AEROSIL © R 812S VV75 | C | A |
| 3 | AEROSIL © R 812S VV90 | C | A |
| 4 | AEROSIL © R 805 | D | A |
| 5 | AEROSIL © R 202 | C | A |
| 6 | 8 parts AEROSIL © R 812S | D | A |
| | VV90 2 parts AEROXIDE © | | |
| | TiO₂ T805 | | |

TABLE 3
Storage stability of cleaning agents/detergents

| | | Storage container | |
|---|---|---|---|
| | | material | |
| Example | Silicon dioxide powder | Glass | Polyethylene |
| 7 | AEROSIL R 812 S | C | A |
| 8 | AEROSIL R 812 S VV 75 | C | A |
| 9 | AEROSIL R 812 S VV 90 | C | A |
| 10 | AEROSIL R 805 | D | A |
| 11 | AEROSIL R 202 | C | A |

1. Process to delay the release of water from a pulverulent preparation comprising cosmetic products, cleaning agents and detergents, wherein the pulverulent preparation contains at least 50 wt. % of water and a hydrophobed silicon dioxide powder, characterised in that the pulverulent preparation s in contact with a hydrophobic material during storage.

2. Process according to claim 1, characterised in that the pulverulent preparation as a cosmetic product contains a substance from the group comprising V light screening filters, dyes and pigments, humectants/skin moisturising agents, deodorising and antiperspirant agents, biogenic substances, insect repellent agents, hydrotropes, anti-dandruff agents, bleaching or skin lightening agents and self-tanning agents, preservatives surfactants/emulsifiers, perfume oils and or plant extracts.

3. Process according to claim 1, characeterised in that the pulverulent preparation as a cleaning a gent or detergent contains hydrogen peroxide.

4. Process according to claim 1, characterised in that the hydrophobic material is polyethylene, polypropylene, polyethylene terephthalate and/or teflon.
